(19) United States
(12) Patent Application Publication
(10) Pub. No.: US 2010/0262028 A1
(43) Pub. Date: Oct. 14, 2010

(54) ECG DATA PROCESSING SYSTEM AND METHOD

(76) Inventor: GuoHui Li, Tianjin (CN)

Correspondence Address: WANG & HO, 66 HILLTOP ROAD, MILLINGTON, NJ, 07946, US

(21) Appl. No.: 12/601,519

(22) PCT Filed: May 22, 2007
(86) PCT No.: PCT/CN07/70039

§ 371 Date: May 10, 2010

(51) Int. Cl. A61B 5/0472

(52) U.S. Cl. 600/515

(57) ABSTRACT

The invention discloses an ECG data processing system and method which process the ECG signal data collected by the system by using software. The method includes the following steps: A/D converting the collected data; performing high-pass filter process for the converted digital signal; performing low-pass filter process for the converted digital signal data; performing the QRS recognition algorithm; and determining whether an abnormal QRS wave occurs; if it does not occur, displaying the waveform; if the abnormal QRS wave occurs, storing the data during a predefined time before the abnormal QRS wave into the storage module, and recording the incident triggering time; and processing the recorded trigger incident. Compared with the prior art, this invention is more accurate for processing ECG signal data. It also provides effective data display, alarm functions, and improved and perfected ECG hardware structure.

### FIELD OF THE INVENTION

The invention relates to electronic medical devices, particularly to an electrocardiograph (ECG) data processing method and system.

### BACKGROUND OF THE INVENTION

Regarding the hardware of the invention, the current ECG device includes an internal lithium battery, a power converter which converts AC 220V to DC 5V, an LCD display, control keys, and body contact electrodes (3 electrodes) with conductive wires. As needed, the ECG device may also include an optical ECG cable and a data communication line connected to a computer USB interface (or wireless data communication interface; e.g. Bluetooth, zigbee, infrared communication interface, etc.). If necessary for a particular application, the ECG device may also be equipped with conductive wires which connect two electrode clamps, one electrode ball and other accessories.

However, in order to further improve and enhance the performance of the ECG device, improvements which are only focused on hardware aspects are insufficient to meet the requirements for expanding its functionality. The development of related technology, especially software technology, has played a very important role in enhancing the functionality of electronic devices, especially ECG data processing and ECG wave display technology.

### SUMMARY OF THE INVENTION

In order to solve the above-mentioned defects, the present invention provides an ECG data processing system and method to enhance the ECG functions in data processing by using software to carry out the ECG signal data collection, processing, and display functions.

The invention provides an ECG data processing system which includes a main control unit 10 that is powered by an ECG power module 70. The input signal of the main control unit is from an amplified ECG signal 101 and the collected results of an ECG signal 102; therein the outputs of the main control unit 10 are respectively coupled to an ECG communication module 20, a clock module 30, a liquid crystal display module 40, a data storage module 50, an alarm module 60, and a keyboard module 70, wherein:

the ECG communication module 20 is used for the ECG data communication;

the clock module 30 is used for recording the system time in the ECG data processing system and the time when an incident happens;

the LCD module 40 is used for displaying the ECG signal as well as other system-related incidents or data information;

the data storage module 50 is used for the system parameters setting as well as the storage of ECG signal data;

the alarm module 60 is used for sounding an alarm in the event of an unusual situation in the system, such as signal abnormalities or battery power abnormalities;

the keyboard module 70 is used for processing keyboard interrupt signals.

The present invention also provides a second technical solution:

An ECG data processing method includes the following steps: firstly, A/D converting the collected data in step 201; performing the high-pass filter process for the converted digital signal in step 202; performing the low-pass filter process for the converted digital signal data in step 203; performing the QRS recognition algorithm in step 204; determining whether the data storage for calculation results is necessary in step 205; if necessary, proceeding to step 208; that is, when the system is abnormal or a user manually stops operation, storing the data during the 12 seconds prior to the abnormal signal or manually-stop operation signal into the data storage module of the system, and recording the incident triggering time; otherwise, if the data storage for calculation results is not necessary in step 205, further determination of whether an abnormal QRS wave occurs in step 206; if it does not occur, then proceeding to step 207, and displaying the waveform by using the LCD module; if the abnormal QRS wave occurs, then performing step 208, and storing the data during a predefined time before the abnormal QRS wave into the storage module and recording the incident triggering time occurs. The incidents are recorded in three different ways:

The first method is: after D/A converting the recorded time, sending the analog signal back to the ECG in step 209; and outputting the analog signal in step 210;

The second method is: reading out the stored ECG incidents and data and performing a more detailed analysis and display in step 211; and outputting the ECG and analysis report in the step 212;

The third method is: transmitting the data to network in step 213; and remotely outputting ECG and analysis reports in step 212.

Compared with the prior art, the benefits of this invention are that it is more accurate to process ECG signal data, it provides effective data display and alarm functions, and the ECG hardware structure is improved and perfected.

### BRIEF DESCRIPTION OF THE DRAWINGS

FIG. 1 shows the structure diagram of the ECG data processing system according to present invention;

FIG. 2 shows the data processing flowchart of the ECG data processing method according to present invention;

FIG. 3 shows the interpolating point method for liquid crystal display location of the ECG data processing system and method according to present invention; and

FIG. 4 shows the schematic position of the electrodes according to present invention.

### DETAILED DESCRIPTION OF EMBODIMENTS OF THE INVENTION

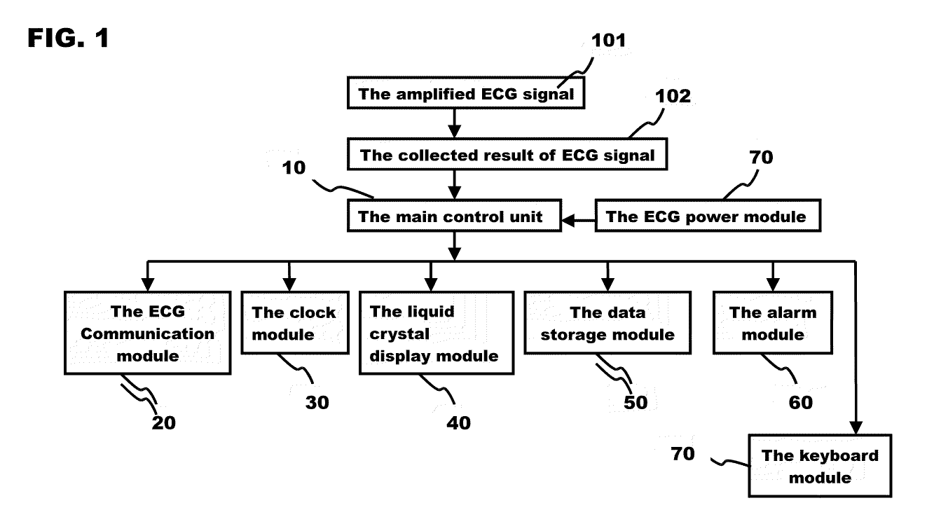

As shown in FIG. 1, the present invention provides an ECG data processing system, which includes a main control unit 10 (CPU). The power of the main control unit is supplied by an ECG power module 70 and an input signal of the main control unit is from an amplified ECG signal 101. The collected result of an ECG signal 102; the outputs of the main control unit are respectively connected to an ECG communication module 20, a clock module 30, a liquid crystal display module 40, a data storage module 50, an alarm module 60, and a keyboard module 70.

Specifically speaking, the ECG communication module 20 is used for the ECG data communication;

The clock module 30 is used for recording the system time in the ECG data processing system and the time when incidents happen;

The liquid crystal display module 40 is used to display the ECG signals as well as other system-related incidents or data information;

The data storage module 50 is used for the system parameters setting as well as the storage of ECG signal data;

The alarm module 60 is used for sounding the alarm in case of unusual situations in the system, such as signal abnormalities or battery power abnormalities;

The keyboard module 70 is used for processing keyboard interrupt signals.

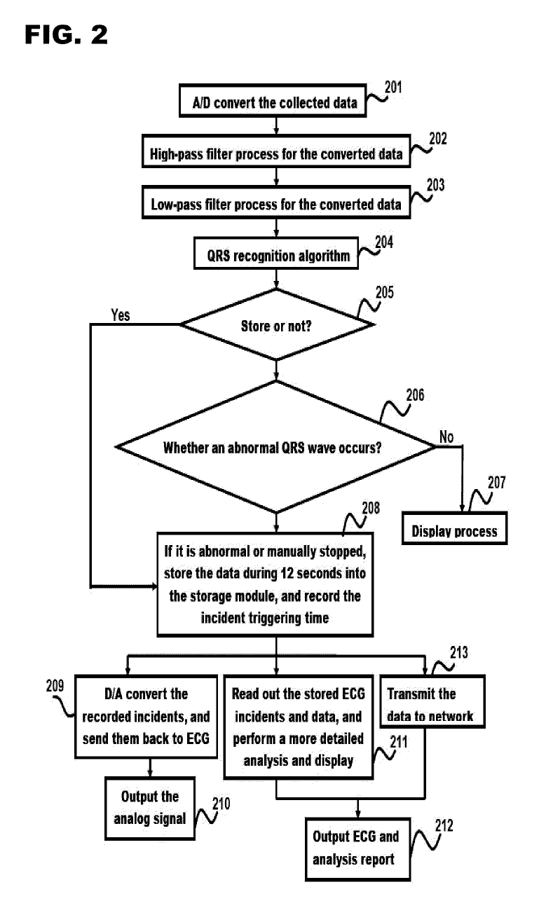

As shown in FIG. 2, it is the ECG data processing method provided by this invention. The method includes the following steps: firstly, A/D converting the collected data in step 201; performing high-pass filter process for the converted digital signal in step 202; performing low-pass filter process for the converted digital signal data in step 203; performing the QRS recognition algorithm in step 204; determining whether the data storage for calculation results is needed at the time in step 205; if needed, proceeding to step 208; that is, when the system is abnormal or a user manually stops operation, storing the data during the 12 second or so prior to the abnormal signal or manually-stop operation signal into data storage module of the system and recording the incident triggering time; otherwise, if the data storage for calculation results is not needed at the time, further determining whether an abnormal QRS wave occurs in step 206; if an abnormal QRS wave does not occur, then proceeding to step 207 and displaying the waveform processing by using the LCD module; if the abnormal QRS wave occurs, then proceeding to step 208, and storing the data during 12 seconds or so prior to the abnormal QRS wave into the storage module and recording the incident triggering time occurs. The incidents are recorded in three different ways:

The first method is: after D/A converting the recorded time, sending the analog signal back to the ECG in step 209 and outputting the analog signal in step 210;

The second method is: reading out the stored ECG incidents and data, and performing a more detailed analysis and display in step 211 and outputting the ECG and analysis report in the step 212;

The third method is: transmitting the data to network in step 211 and remotely outputting ECG and analysis reports in step 212.

The above-mentioned technical solutions of this invention will be further described through following embodiments.

Display the ECG data after collection

According to the medical requirements and the ECG design requirements, about 500 or 1000 sampling points are collected. The image showing rate may be 12.5 mm/Sec, 25 mm/Sec, 50 mm/Sec, or 100 mm/Sec, and the amplitude may be 5 mm/mV, 10 mm/mV, 20 mm/mV, or 40 mm/mV. The collected points per second and their values are certain, therefore requiring a certain algorithm to meet the functional requirements of the ECG device.

In order to understand the program easily, a few constants and variables are declared firstly.

(PASCAL) const aArrayTSS: array[1.4] of string=('12.5 mm/Sec', ‘25 mm/Sec’, ‘50 mm/Sec’, ‘100 mm/Sec’); (display speed character array)

aArrayTSI: array[1.4] of Single=(12.5, 25, 50, 100); (display speed value array)

aArrayVSS: array[1.4] of string=('5 mm/mV', ‘10 mm/mV’, ‘20 mm/mV’, ‘40 mm/mV’); (display amplitude character array)

aArrayVSI: array[1.4] of Single=(5, 10, 20, 40); (display amplitude value array)

SampleHZ: Integer=512; //Sampling frequency

Xppsi: the number of sampling points for each pixel

xppmm: the number of pixels per millimeter in X direction. The number of pixels per millimeter in X direction is the same as that in Y direction in principle. If necessary, it can also add yppmm, e.g. the number of pixels per millimeter in Y direction.

tscale: the defined display speed per second (mm/Sec)

vscale: the defined display amplitude of each mV (mm/mV)

2. The number of pixels for each sampling point are calculated by formulation: xppsi=xppmm * tscale/SampleHZ;

3. In order to ensure that the collected data can be displayed according to the required amplitude, the collected data must be transformed into corresponding raw voltage values and then multiplied by the value of the currently displayed vsacle value, resulting in the display amplitude of the current collected points. The collected voltage value * xppmm * tscale is the pixel value of the display amplitude of the current collected points.

4. When drawing a point, the position order number of the current sampling point must first be known. This sampling point is then multiplied by xppsi to determine the value of the display pixel location of this sampling point. This value is then compared with the location of the last display pixel.

1) If it is in the same display position as the last sampling point (pixel number), it is not necessary to display the point, but if the value of a pixel displayed is relatively far away from the baseline, it should be recorded(thus without losing the characteristic values of the ECG or graphic distortion).

2) If it is greater than the display position of the last sampling point (pixels), the point is connected with another point, e.g. to draw the display waveform.

5) If there is no function used to draw a line for liquid crystal display (only function for drawing points), it is necessary to interpolate some points between two points with a large distance between them so that the figure will not be displayed incoherently.

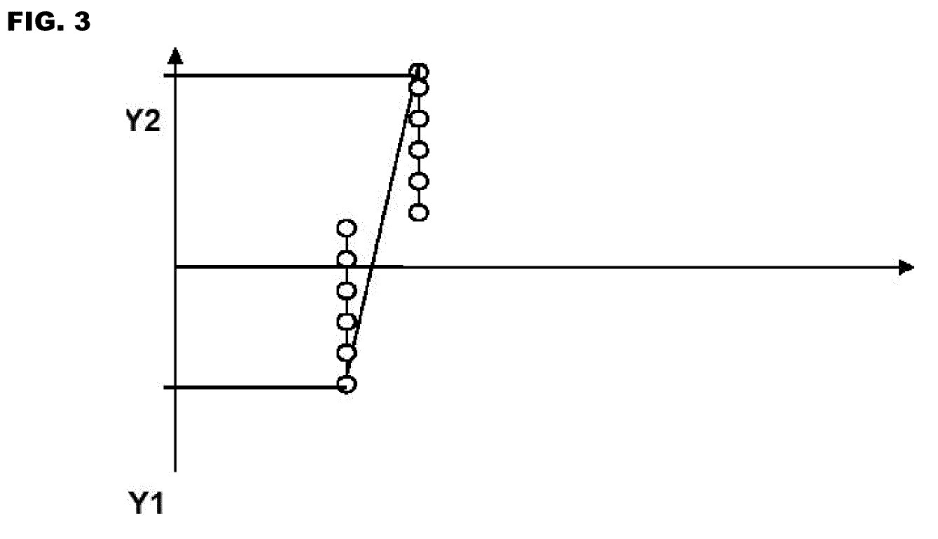

6. The diagram of the interpolation point method is shown in FIG. 3: (zoom in)

In the above figure, it is assumed that two points in the X-direction are the most closed points, e.g. only one pixel is between the two points in the X direction. If the distance between two display pixel points in the Y direction is farther, interpolation must be performed in order to make it look smoother. By setting the mid-point of Y1 and Y2 as the boundary, the pixel number value from Y1 to the mid-point is interpolated at the X1, and the pixel number value from Y2 to the mid-point is interpolated at the X2.

7. It is necessary to set a collected data buffer of at least 2 seconds in order to save the collected data, so that the first 2 seconds of data could be used when the data is stored.

8. The source code is shown relatively complete.

In the ECG device according to this invention, two or three electrodes can be set for ECG conducting and monitoring. The relationship between the electrodes for ECG conducting and monitoring commonly used is shown in following Table 1:

| | Amperemeter | Amperemeter |
|---|---|---|
| Electrode | Positive end | negative end |
| Standard Conducting: | Top left arm | Top right arm |
| II(L2) | | |
| Standard Conducting: | Bottom left arm | Top right arm |
| II(L2) | | |
| Standard Conducting: | Bottom left arm | Top left arm |
| III(L2) | | |

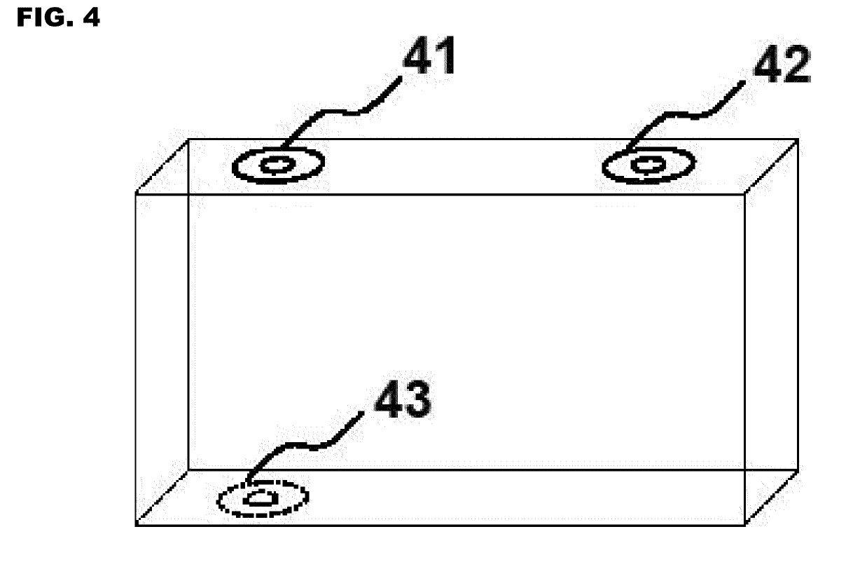

The ECG electrodes' location is shown in FIG. 4. Among them, 41 is a current positive end, 42 is a current negative end, and 43 is a reference electrode connected to grounding.

### The ECG Heart Rate Calculation

1. With regard to the calculation method of heart rate, it can be roughly understood as follows:

According to the collected data, filter=(t0=v [0])+4*t1+6*t2+4*t3′t4-t5 −4*t6−6*t7−4*t8−t9.

Noise signal can be omitted from the collected data and the changing trend in the last few sampling points, e.g. the slope of the collected data, can be calculated. According to the obtained slope and the threshold of R-wave slope, the location of R-wave can be determined. Heart rate is then calculated based on the number of R-waves (heartbeats).

2. In order to ensure the accuracy of heart rate and avoid errors from disturbances, it is necessary to calculate the heart rate once for every collected 5 R-waves.

3. Relatively Completed R-Wave Detection Algorithm.

### Data Storage for ECG

1. There are four possible storage solutions, i.e. 8 seconds, 10 seconds, 16 seconds, and 20 seconds.

2. The data during two seconds before the current time and -2 seconds subsequent to specified storage time should be stored when storage button is clicked.

### Data Reproduction of ECG

1. There are two reproduction speeds: 50 mm/Sec & 100 mm/Sec.

2. There are four display amplitudes during reproduction, the same as that during collection.

What is claimed is:

1. An ECG data processing system, comprising a main control unit (10) whose power is supplied by an ECG power module (70), wherein an input signal of the main control unit is from an amplified ECG signal (101) and from collected result of an ECG signal (102); wherein the outputs of the main control unit (10) are respectively connected to an ECG communication module (20), a clock module (30), a liquid crystal display module (40), a data storage module (50), an alarm module (60), and a keyboard module (70), wherein the ECG communication module (20) is used for the ECG data communication; the clock module (30) is used for recording the system time in the ECG data processing system and time when an incident happens; the liquid crystal display module (40) is used for displaying the ECG signal as well as other system-related incidents or data information; the data storage module (50) is used for the system parameters setting, as well as the storage of ECG signal data; the alarm module (60) is used for sounding an alarm in the event of unusual situations in the system, such as signal abnormalities or battery power abnormalities; and the keyboard module (70) is used for processing keyboard interrupt signals.

2. The ECG data processing system as claimed in claim 1, wherein the LCD display module (40) displays interpolation points between two display positions with a large distance between them.

3. An ECG data processing method which processes the ECG signal collected by a system by using software, includes following steps:
A/D converting the collected data;
performing high-pass filter process for the converted digital signal;
performing low-pass filter process for the converted digital signal data;
performing the QRS recognition algorithm;
determining whether the data storage for calculation results is necessary;
if necessary, recording the incident triggering time;
otherwise, determining whether an abnormal QRS wave occurs or not;
if it does not occur, displaying the waveform processing;
if the abnormal QRS wave occurs, storing the data during the 12 seconds prior to the abnormal QRS wave into the storage module, to recording the incident triggering time; and
processing the recorded trigger incident.

4. The ECG data processing method as claimed in claim 3, wherein the step of processing the recorded trigger incident further includes three different ways:
the 1st method is:
sending the analog signal back to the ECG after D/A converting the recorded time; and outputting the analog signal;
the 2nd method is:
reading out the stored ECG incidents and data, and performing a more detailed analysis and display; and outputting the ECG and analysis report;
the 3rd method is:
transmitting the data to network; and remotely outputting the ECG and analysis report.

5. The ECG data processing method as claimed in claim 3, wherein 256 sampling points are collected per second during the collecting ECG samples.

6. The ECG data processing method as claimed in claim 3, wherein a collecting data buffer of at least 2 seconds is set for the sample data.
